(19) United States
(12) Patent Application Publication
(10) Pub. No.: US 2016/0299111 A1
(43) Pub. Date: Oct. 13, 2016

(54) SYSTEMS AND METHODS FOR ASSESSING ENVIRONMENTAL CONDITIONS

(71) Applicant: Caterpillar Inc., Peoria, IL (US)

(72) Inventors: Maria Cristina Herrera de KONTZ, Chillicothe, IL (US); Aloke Jude MASCARENHAS, Peoria, IL (US); Matthew Edward KONTZ, Chillicothe, IL (US); Jeffrey Lee KUEHN, Germantown Hills, IL (US)

(73) Assignee: Caterpillar Inc., Peoria, IL (US)

(21) Appl. No.: 14/680,803

(22) Filed: Apr. 7, 2015

(51) Int. Cl. G01N 33/00; G01W 1/00

(52) U.S. Cl. CPC G01N 33/0075; G01W 1/00; G01N 33/0062

(57) ABSTRACT

Systems and methods are disclosed for assessing environmental conditions. According to certain embodiments, environmental data is received from a machine traveling through a first location and a second location. The environmental data includes first environmental data associated with the first location and second environmental data associated with the second location. Environmental conditions associated with a third location may be interpolated based on the first and second environmental data.

### TECHNICAL FIELD

The present disclosure generally relates to systems and methods for assessing environmental conditions and, more particularly, to interpolating environmental conditions at a particular location based on environmental data from other locations.

### BACKGROUND

Work environments associated with certain industries, such as the mining and construction industries, are susceptible to undesirable dust conditions. For example, worksites associated with mining, excavation, construction, landfills, and material stockpiles may be particularly susceptible to dust due to the nature of the materials composing the worksite surface. Elevated dust conditions may reduce the efficiency at a worksite. For example, dust may impair visibility, interfere with operations on the site, and require increased equipment maintenance and cleaning. Moreover, excessive dust conditions may compromise the comfort, health, and safety of worksite personnel.

To address elevated dust conditions at a worksite, haul roads and other surfaces of a worksite may be watered, for example, by a truck equipped with one or more sprayers. Excessive watering, however, may create slick conditions, which may threaten the safety of operators of machines that drive through the worksite. Thus, the watering process must be carefully managed to ensure that the right amount of water is distributed to the appropriate areas within a worksite. Existing systems for controlling dust conditions rely largely on visual observation by worksite operators. For example, an operator may notify the appropriate worksite personnel that drivers are experiencing difficulty driving in a certain area of the worksite because of low visibility due to excessive dust. Moreover, while some systems rely on automatic detection of dust conditions by one or more sensors to identify a need for watering in a given area, these systems only address needs after they have arisen and are unable to predict a need for watering and, thus, prevent excessive dust from accumulating.

One system for delivering fluid in a worksite is described in U.S. Pat. No. 8,360,343. The '343 patent describes a fluid delivery machine for delivering water to areas of a worksite experiencing excessive dust conditions. According to the '343 patent, the fluid delivery machine receives fluid delivery mission instructions from a site computing system. The mission instructions identify locations at the worksite that need to be watered, as well as an amount of water that should be delivered to those locations. Based on the instructions, the fluid delivery machine may deliver water to the identified locations, thereby remedying the excessive dust conditions.

Although the '343 patent discloses techniques for automating the treatment of excessive dust conditions at a work site, the '343 patent does not describe how to predict these conditions before they occur and distribute water or other fluids throughout the worksite based on predicted needs. Moreover, the '343 patent discloses sensors for detecting dust conditions within a worksite, but does not describe how to extrapolate the information detected by these sensors to determine watering needs for areas where measurements have not been performed.

The present disclosure is directed to overcoming one or more of the problems set forth above and/or other problems in the art.

### SUMMARY OF THE INVENTION

In one aspect, the present disclosure is directed to a method for assessing environmental conditions. The method is performed by one or more processors and includes receiving environmental data from a machine traveling through a first location and a second location. The environmental data includes first environmental data associated with the first location and second environmental data associated with the second location. The method also includes interpolating environmental conditions associated with a third location based on the first and second environmental data.

In another aspect, the present disclosure is directed to a non-transitory computer-readable storage medium storing instructions for assessing environmental conditions. The instructions cause the at least one processor to receive environmental data from a machine traveling through a first location and a second location. The environmental data includes first environmental data associated with the first location and second environmental data associated with the second location. The operations further include interpolating environmental conditions associated with a third location based on the first and second environmental data.

In yet another aspect, the present disclosure is directed to a system for assessing environmental conditions, including a memory that stores a set of instructions and at least one processor in communication with the memory and configured to execute the set of instructions to perform certain steps. The processor is configured to receive environmental data from a machine traveling through a first location and a second location. The environmental data includes first environmental data associated with the first location and second environmental data associated with the second location. The processor is also configured to interpolate environmental conditions associated with a third location based on the first and second environmental data.

### BRIEF DESCRIPTION OF THE DRAWINGS

FIG. 1 is a representation of an exemplary worksite on which the disclosed processes may be employed;

FIG. 2 is a representation of an exemplary fluid delivery machine, consistent with the disclosed embodiments;

FIG. 3 is a representation of an exemplary fluid delivery control system associated with the fluid delivery machine of FIG. 2, consistent with the disclosed embodiments;

FIG. 4 is a representation of an exemplary flow control system associated with the fluid delivery control system of FIG. 3, consistent with the disclosed embodiments;

FIG. 5 is a representation of an exemplary weather station, consistent with the disclosed embodiments;

FIG. 6 is a flow chart illustrating an exemplary disclosed method for identifying undue dust conditions, in accordance with certain embodiments;

FIG. 7 is a flow chart illustrating an exemplary disclosed method for assessing environmental conditions, in accordance with certain embodiments; and

FIG. 8 is a flow chart illustrating an exemplary disclosed method for remedying undue dust conditions, in accordance with certain embodiments.

### DETAILED DESCRIPTION

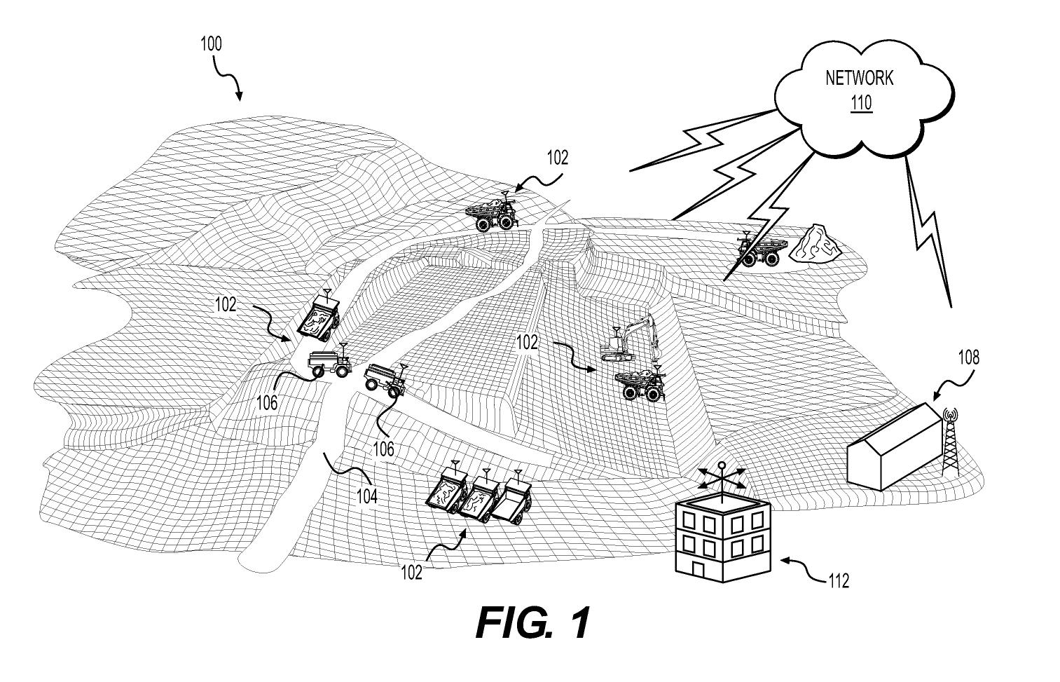

FIG. 1 illustrates an exemplary worksite 100 at which the disclosed processes may be employed. In one environment, worksite 100 may comprise a surface mine site at which mining operations generate significant dust levels that create difficult conditions for worksite personnel. For example, the dust may impair visibility, reduce air quality, require frequent equipment maintenance and cleaning, or otherwise hinder operations. It is to be appreciated, however, that worksite 100 may alternatively include a construction site, a landfill, an underground worksite, or any other type of worksite at which undesired dust conditions may arise.

As shown in FIG. 1, a variety of mobile machines 102 may operate on worksite 100. Mobile machines 102 may include any combination of autonomous (i.e., unmanned) machines, semi-autonomous machines, or operator-controlled machines. Mobile machines 102 may include, for example, off-highway haul trucks, articulated trucks, excavators, loaders, dozers, scrapers, or other types of earth-working machines for excavating or handling material associated with worksite 100. In connection with various work operations, mobile machines 102 may travel along haul roads 104 or other paths between excavation locations, dumping areas, and other destinations on worksite 100. Aside from earth-working machines and other such heavy equipment, mobile machines 102 may also include one or more fluid delivery machines 106. Fluid delivery machines 106 may be configured to travel worksite 100 along haul roads 104 and to deliver fluid (e.g., water and/or other dust suppressant) to the surface of worksite 100 to control dust levels.

Mobile machines 102 may include various indicators and sensors for detecting and/or representing conditions associated with the internal and external environments of mobile machines 102. For example, mobile machines 102 may include a traction control system (TCS), anti-lock braking system (ABS), and dynamic stability control (DSC) system. TCS is designed to prevent loss of traction for one or more wheels of a mobile machine. For example, if TCS detects that one or more wheels is spinning significantly faster than another, it may cause brakes to be applied to the one or more wheels that are spinning with lessened traction. ABS is activated to allow the wheels to maintain tractive contact with the ground while braking, such that the wheels do not lock up or skid across the surface. DSC improves a mobile machine's stability by detecting and reducing loss of traction, such as by selectively applying breaks to steer the mobile machine where the operator intends to go.

Each of these systems may be associated with an indicator (i.e., a TCS indicator, an ABS indicator, a DSC indicator) to represent whether the system is active in the mobile machine. The activation of TCS, ABS, or DSC may indicate a change in ground conditions. For example, TCS or DSC may be activated when there is a loss of traction in one or more wheels of the mobile machine. A loss of traction may suggest that the ground surface is loose (e.g., too dry) or overly saturated (e.g., too wet). ABS may be activated when surface conditions affect the ability of a mobile machine to brake safely. The presence of conditions that cause the ABS to activate may also indicate that the ground surface is either too dry or too wet.

Mobile machines 102 may include one or more dust sensors. A dust sensor may include any device configured to determine a dust condition or a dust level of the air. For example, a dust sensor may collect an air sample, pass a constant-intensity light beam from a light source through the air and toward a light sensor, and measure the magnitude of light transmission interference between the light source and the light sensor. A dust sensor may determine the concentration of the dust in the air based on the magnitude of the interference. It should be appreciated that alternative or additional types of dust monitoring devices or methods known in the art may be used.

Mobile machines 102 may also include one or more moisture sensors. A moisture sensor may include any device configured to determine the moisture content (e.g., volumetric water content) of the surface of worksite 100. For example, a mobile machine may place a moisture sensor in contact with the surface to sense a moisture content of the worksite surface.

Mobile machines 102 may further include a machine location device. The machine location device may include any device configured to determine a real-time location of the machine. The machine location device may receive and analyze high-frequency, low-power radio or laser signals from multiple locations to triangulate a relative location (e.g., in latitude and longitude) of the machine. For example, the machine location device may comprise an electronic Global Positioning System (GPS) receiver, a Global Navigation Satellite Systems (GNSS) receiver, or another type of receiver configured to receive signals from one or more satellites and to determine the location of the machine based on the signals. Alternatively or additionally, the machine location device may comprise a local radio or laser system configured to receive a signal from one or more transmission stations, and to determine a relative 2-D or 3-D location of the machine with respect to known locations of the transmission stations. Alternatively or additionally, the machine location device may include an Inertial Reference Unit (IRU), an odometric or dead-reckoning positioning device, or another known locating device operable to receive or determine a relative 2-D or 3-D location of the machine in real-time. The machine location device may, in real-time or periodically, generate and communicate to other mobile machines 102 and/or a worksite control system 108 a signal indicative of the location of the machine on worksite 100 (e.g., in latitude and longitude) for use in the disclosed fluid delivery processes, as discussed below.

In connection with their various operations, mobile machines 102 may communicate with one another, and with worksite control system 108, over an electronic network 110 (e.g., comprising the Internet). For example, mobile machines 102 may communicate location information to one another over network 110, so that machine operators will know what areas of worksite 100 are being worked by other operators. Mobile machines 102 may also communicate the status of the ABS, DSC, and TCS indicators (e.g., active or inactive) and measurements of the dust and moisture sensors, along with associated location information (i.e., a GPS location associated with a particular measurement or indicator status), to worksite control system 108, which may analyze this information to determine dust, traffic, and/or other environmental conditions at various areas of worksite 100.

Worksite control system 108 may include one or more server systems, databases, and/or computing systems configured to receive data from entities over a network and process and/or store the information. For example, worksite control system 108 may receive data over network 110 from mobile machines 102, fluid delivery machines 106, a weather station 112, and third-party sources, store and/or process the data, and send the processed data over network 110 to fluid delivery machines 106 and other data consumers. In one embodiment, worksite control system 108 may include a processing engine and one or more databases for storing and processing the data.

As discussed above, worksite control system 108 may receive data from mobile machines 102, fluid delivery machines 106, weather station 112, and third-party sources. For example, worksite control system may receive machine data from mobile machines 102 that describes the operation of the machines and/or environmental conditions observed as mobile machines 102 operate on the worksite. In one embodiment, the received machine data may include data describing the state of the machine's anti-lock braking system (ABS), traction control system (TCS), and/or dynamic stability control (DSC) system. The received machine data may also include data collected by one or more dust sensors (e.g., dust measurements) and/or moisture sensors (e.g., moisture values) located on the mobile machine 102.

Worksite control system 108 may receive data from fluid delivery machines 106 describing the delivery of fluids by fluid delivery machines to various locations within worksite 100. For example, worksite control system 108 may receive data regarding the route traveled by the fluid delivery machine (e.g., a list of geographic locations within the worksite), a rate of flow of fluid at each location, a spray nozzle setting used at each location, and a speed of the fluid delivery machine at each location.

Worksite control system 108 may further receive data from weather station 112 describing current, historical, or forecasted weather conditions of worksite 100. For example, worksite control system 108 may receive data regarding ambient temperature, solar radiation, atmospheric pressure, relative humidity, wind speed and direction, and precipitation at worksite 100. Worksite control system 108 may also receive data regarding current, historical, or forecasted weather conditions from one or more third-party weather sources. In addition to weather information, worksite control system 108 may receive other data from third-party sources, such as traffic data from third-party mapping and/or traffic sources.

The data received and stored by worksite control system 108 is stored (e.g., in a database or other memory) and processed (e.g., by a processing engine). For example, worksite control system 108 may process the received data to analyze and interpolate environmental conditions and generate one or more parametric dust models, fluid delivery requirements, and fluid delivery plans. The processed data (e.g., the fluid delivery plans) may be transmitted over network 110 to fluid delivery machines 106 to guide delivery of fluids throughout worksite 100.

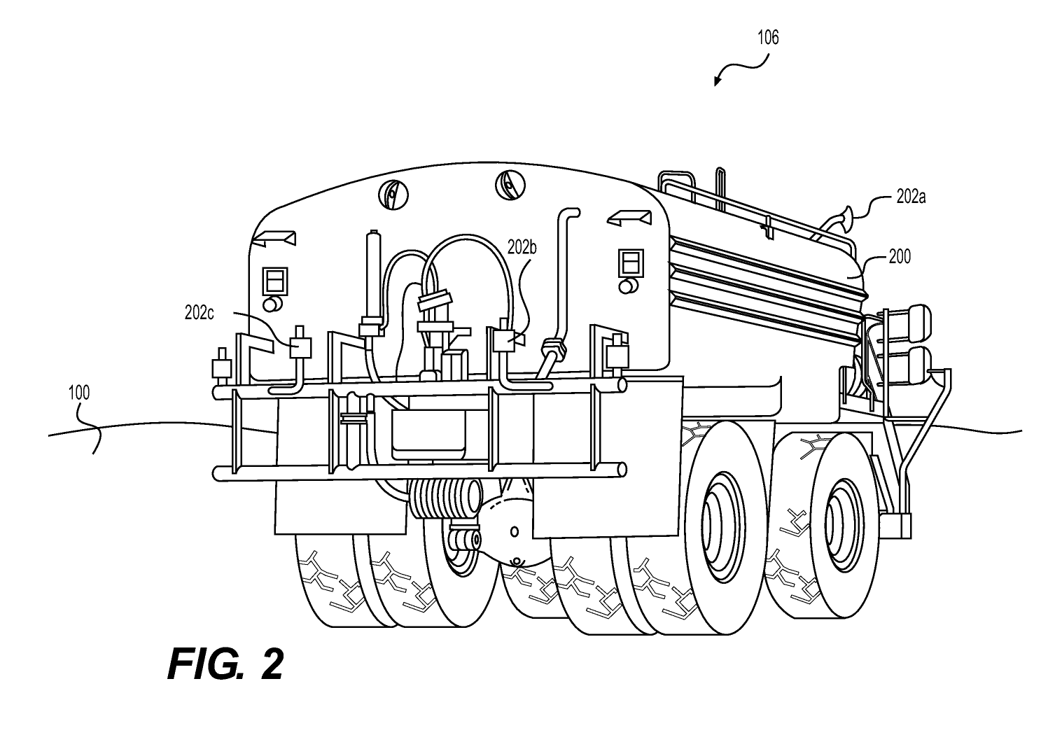

FIG. 2 illustrates an exemplary fluid delivery machine 106, consistent with the disclosed embodiments. In one embodiment, fluid delivery machine 106 may be an off-highway truck converted for use to deliver fluid. For example, fluid delivery machine 106 may be fitted with, among other things, a fluid tank 200 configured to store fluid (e.g., water); a variety of piping, hoses, pumps, and valves; and one or more spray heads 202 (e.g., 202a, 202b, 202c) configured to spray the fluid onto the surface of worksite 100 as fluid delivery machine 106 travels about worksite 100, thereby controlling dust conditions on worksite 100. It is to be appreciated that the disclosed fluid delivery machine 106 may alternatively comprise another type of mobile machine set up to distribute water or other fluids in a wide variety of applications. For example, fluid delivery machine 106 may embody a tractor towing a trailer equipped with a tank and configured to distribute chemicals, water, or other materials (e.g., pesticide, fertilizer, etc.) in an agricultural setting; an on-highway truck configured to spray a saline solution on roads, runways, or parking lots to melt snow and ice; or another type of vehicle configured to deliver fluid in another environment.

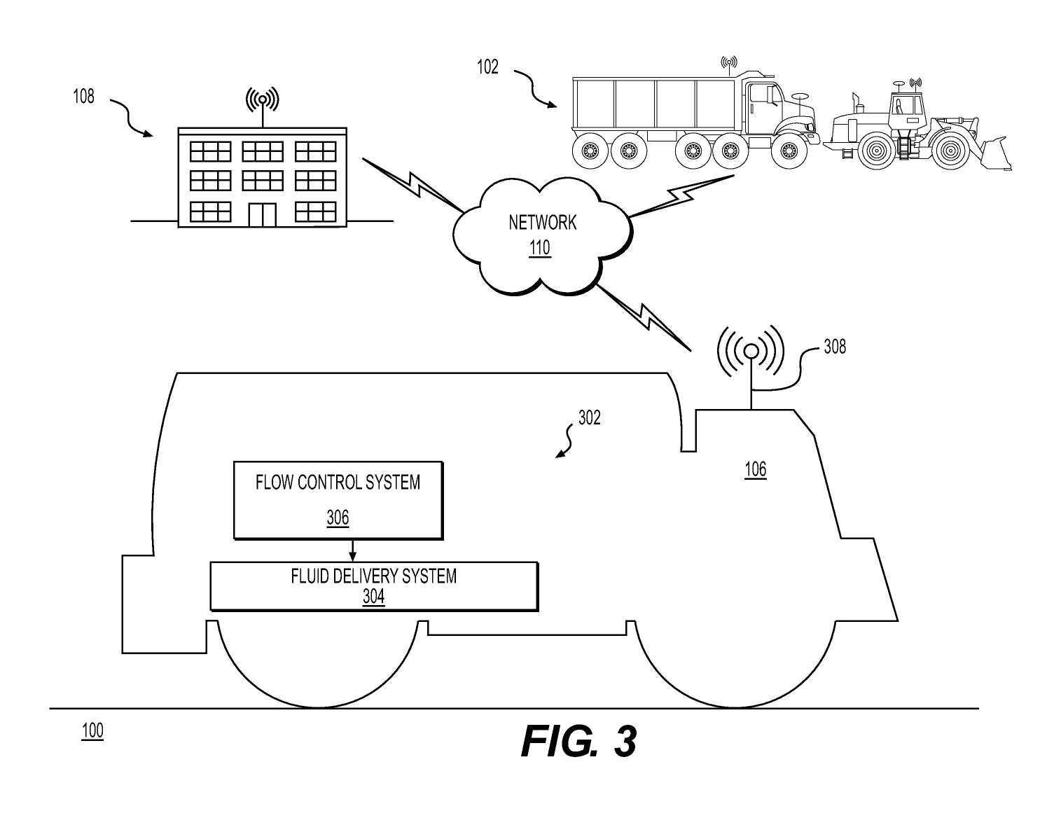

FIG. 3 illustrates an exemplary fluid delivery control system 302, consistent with the disclosed embodiments. For purposes of illustration, fluid delivery control system 302 is described as applied to fluid delivery machine 106 (e.g., an off-highway truck set up for use as a water truck at a mining or construction site). As noted above, however, fluid delivery control system 302 may be applicable in a variety of other scenarios. As shown in FIG. 3, fluid delivery control system 302 may include a fluid delivery system 304 and a flow control system 306 for distributing fluid, such as water, on the surface of worksite 100 to alleviate dust conditions. Onboard fluid delivery control system 302 may communicate with worksite control system 108 and with other mobile machines 102 via network 110 using a communication device 308 (e.g., an antenna).

Fluid delivery system 304 may be configured to distribute fluid (e.g., water or other dust suppressant) on the surface of worksite 100 at a rate commanded by flow control system 306. Flow control system 306 may be configured to determine an appropriate fluid delivery rate (e.g., in liters per square meter per hour) and spray width or distribution under the circumstances, and to output a desired flow rate signal commanding fluid delivery system 304 to output fluid on the worksite surface at the determined rate and distribution.

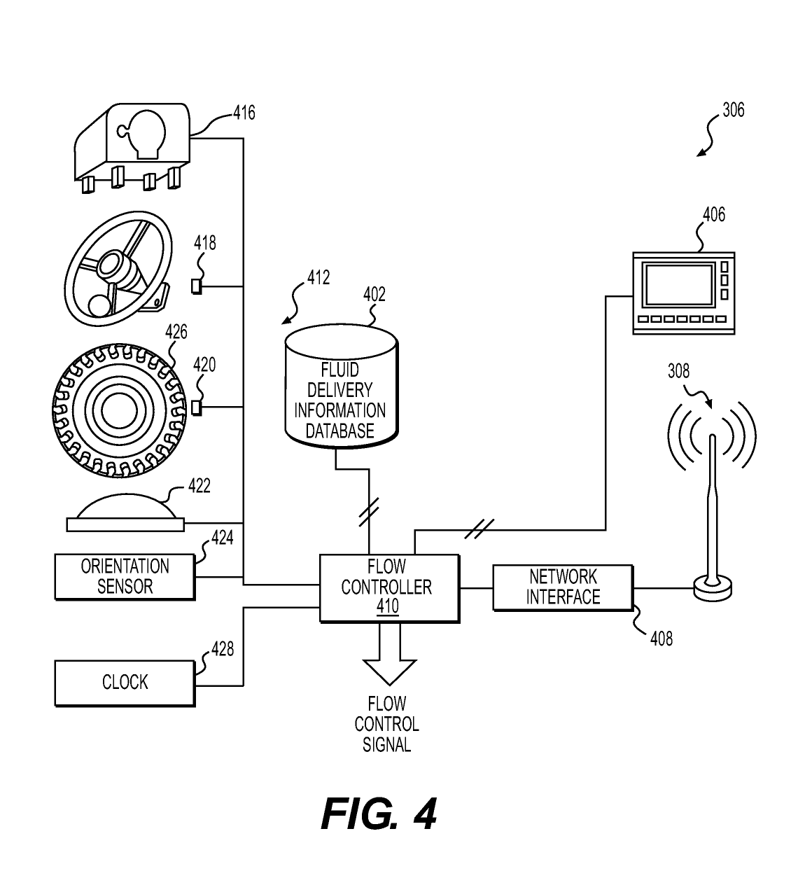

FIG. 4 illustrates a representation of flow control system 306 in greater detail. As shown, flow control system 306 may include a sensing system 412, a fluid delivery information database 402, an operator interface 406, and a network interface 408 in communication with a flow controller 410. Flow control system 306 may include a variety of devices for sensing different parameters in connection with the disclosed fluid delivery processes.

Sensing system 412 may include a variety of sensing devices for sensing different operational parameters of fluid delivery machine 106 in connection with the disclosed fluid delivery processes. For example, sensing system 412 may include a machine vision device 416, a steering angle sensor 418, a traction device speed sensor 420, a machine location device 422, and a machine orientation sensor 424.

Machine vision device 416 may include a device positioned on fluid delivery machine 106 and configured to detect a range and a direction to points on the surface of worksite 100 (e.g., objects) within a field of view of machine vision device 416. Machine vision device 416 may comprise a LIDAR (light detection and ranging) device, a RADAR, (radio detection and ranging) device, a SONAR (sound navigation and ranging) device, a camera device, or any other type of device that may detect a range and a direction to points on the surface of worksite 100. Machine vision device 416 may, in real-time or periodically, generate and communicate to flow controller 410 a signal indicative of the range and the direction to the points on the surface of worksite 100 for use in the disclosed fluid delivery processes, as discussed below. In one aspect, as fluid delivery machine 106 travels about worksite 100, machine vision device 416 may be used to detect objects on the surface of worksite (e.g., other mobile machines 102, worksite personnel, worksite infrastructure, etc.) to determine whether fluid delivery should be halted or modified. For example, it may be desirable to halt or modify fluid delivery when a service vehicle, another machine 102, equipment, or a worker is detected nearby fluid delivery machine 106 to prevent such objects from being sprayed with fluid.

Moreover, machine vision device 416 may be used to monitor spray heads 202 to determine whether fluid delivery system 304 is functioning properly. For example, one or more machine vision devices 416 may be positioned to monitor the fluid sprayed from spray heads 202. If machine vision device 416 detects less than an expected amount of fluid sprayed from spray heads 202 (e.g., no fluid is sprayed from a spray head 202 when the spray head should be spraying some fluid), it may be determined that fluid delivery system 304 is not functioning properly. Based on such a determination, one or more corrective actions may then be taken. For example, fluid delivery system 304 may enter a diagnostic mode whereby spray heads 202 or other elements of fluid delivery system 304 are purged (e.g., to remove a clog).

Steering angle sensor 418 may include any device configured to sense or otherwise determine a steering angle of fluid delivery machine 106. Steering angle sensor 418 may, in real-time or periodically, generate and communicate to flow controller 410 a signal indicative of the determined steering angle for use in the disclosed fluid delivery processes, as discussed below. For example, it may be desirable to reduce or modify fluid delivery when fluid delivery machine 106 is traveling through a curved section of haul road 104.

Traction device speed sensor 420 may include any device configured to determine the speed of one or more traction devices 426 (e.g., wheels) of fluid delivery machine 106. Traction device speed sensor 420 may, in real-time or periodically, generate and communicate to flow controller 410 a signal indicative of the determined speed of traction devices 426 for use in the disclosed fluid delivery processes, as discussed below.

Machine location device 422 may include any device configured to determine a real-time location of fluid delivery machine 106 on worksite 100. Location device 422 may receive and analyze high-frequency, low-power radio or laser signals from multiple locations to triangulate a relative location (e.g., in latitude and longitude) of fluid delivery machine 106. For example, location device 422 may comprise an electronic Global Positioning System (GPS) receiver, a Global Navigation Satellite Systems (GNSS) receiver, or another type of receiver configured to receive signals from one or more satellites and to determine the location of fluid delivery machine 106 based on the signals. Alternatively or additionally, machine location device 422 may comprise a local radio or laser system configured to receive a signal from one or more transmission stations, and to determine a relative 2-D or 3-D location of fluid delivery machine 106 with respect to known locations of the transmission stations. Alternatively or additionally, location device 422 may include an Inertial Reference Unit (IRU), an odometric or dead-reckoning positioning device, or another known locating device operable to receive or determine a relative 2-D or 3-D location of fluid delivery machine 106 in real-time. Location device 422 may, in real-time or periodically, generate and communicate to flow controller 410 a signal indicative of the location of fluid delivery machine 106 on worksite 100 (e.g., in latitude and longitude) for use in the disclosed fluid delivery processes, as discussed below.

Machine orientation sensor 424 may include any device configured to determine a heading and inclination (i.e., orientation) of fluid delivery machine 106 on the surface of worksite 100. For example, orientation sensor 424 may include a laser-level sensor, a tilt sensor, inclinometer, a radio direction finder, a gyrocompass, a fluxgate compass, or another known device operable to determine a relative pitch, yaw, and/or roll of fluid delivery machine 106 as it travels about worksite 100. It is to be appreciated that the combination of the components of pitch, yaw, and roll of fluid delivery machine 106 may indicate the relative slope or inclination of the surface of worksite 100 at the location of fluid delivery machine 106. Orientation sensor 424 may, in real-time or periodically, generate and communicate to flow controller 410 a signal indicative of a heading and inclination of fluid delivery machine 106 for use in the disclosed fluid delivery processes, as discussed below.

Flow control system 306 may also include a clock 428 for determining the current time of day and date. Clock 428 may periodically communicate a signal indicative of the time of day and date to flow controller 410 for use in the disclosed fluid delivery processes, discussed below. In one aspect, the time and date may be appended to or otherwise included with the signals associated with the other sensors discussed above.

Fluid delivery information database 402 may contain information enabling fluid delivery machine 106 to identify locations on worksite 100 at which to deliver fluid, and to determine an appropriate fluid delivery rate at the locations. For example, fluid delivery information database 402 may receive and store one or more fluid delivery plans, including one or more routes or schedules for fluid delivery, from worksite control system 108.

Operator interface 406 may include a monitor, a touch-screen, a keypad, a control panel, a keyboard, a joystick, a lever, pedal, a wheel, or any other device known in the art for receiving input from or providing output to an operator. In connection with the disclosed fluid delivery processes, operator interface 406 may receive input from a machine operator, and may generate and communicate corresponding command signals to flow controller 410. Operator interface 406 may also display information to the machine operator based on signals received from flow controller 410.

Network interface 408 may include any hardware or software for sending and receiving data over network 110. For example, network interface 408 may include a modem, an Ethernet communication device, a fiber optic communication device, a cellular communication device, an infrared communication device, a satellite communication device, and/or any other network communication device capable of transmitting and receiving data over network 110. Accordingly, network interface 408 may be configured to communicate using satellite, cellular, infrared, radio, or other types of wireless communication signals.

Flow controller 410 may include means for monitoring, recording, storing, indexing, processing, or communicating information in connection with the disclosed fluid delivery processes. Flow controller 410 may include a memory, a secondary data storage device (e.g., a magnetic or optical disc drive), a processor (e.g., a CPU), or any other components for running programs for performing the disclosed functions of flow control system 306. Various other circuits may be associated with flow controller 410, such as power supply circuitry, signal conditioning circuitry, data acquisition circuitry, signal output circuitry, signal amplification circuitry, and other types of circuitry known in the art. Flow controller 410 may receive the signals from the various sensors of sensing system 412, and may store the values associated with the sensed parameters in memory for use in subsequent processing.

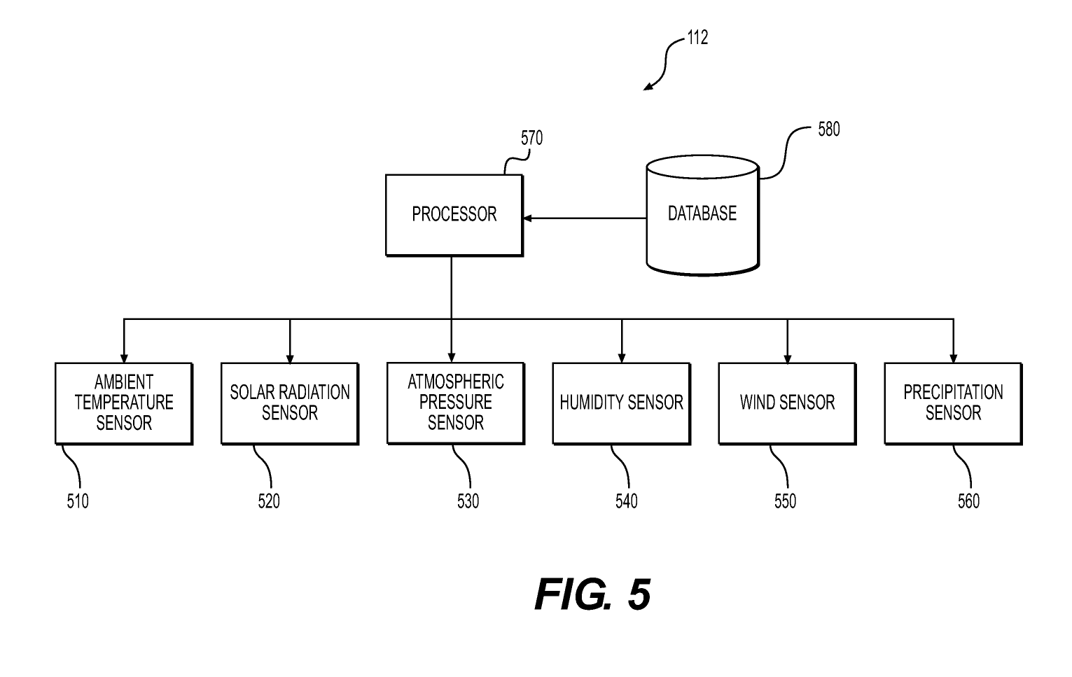

FIG. 5 illustrates a representation of weather station 112 in greater detail. Weather station 112 may include a variety of sensing devices for sensing different weather or environmental parameters associated with worksite 100, in connection with the disclosed systems and methods. For example, weather station 112 may include an ambient temperature sensor 510, a solar radiation sensor 520, an atmospheric pressure sensor 530, a humidity sensor 540, a wind sensor 550, a precipitation sensor 560, a processor 570, and a database 580.

Ambient temperature sensor 510 may include any device (e.g., positioned on fluid delivery machine 106 or at a stationary location on or near worksite 100) configured to sense an ambient temperature of worksite 100. For example, ambient temperature sensor 510 may comprise an analog or digital temperature sensor, a resistance temperature detector (RTD), a thermocouple, a thermowell, or any other type of temperature sensor known in the art. Ambient temperature sensor 510 may, in real-time or periodically, generate and communicate to processor 570 a signal indicative of a value of the sensed ambient temperature (e.g., in degrees Celsius, Fahrenheit, or Kelvin) of worksite 100 for storage in database 580 and/or transmission to worksite control system 108.

Solar radiation sensor 520 may include any device (e.g., positioned on fluid delivery machine 106 or at a stationary location on or near worksite 100) configured to sense an intensity of solar radiation at worksite 100. For example, solar radiation sensor 520 may comprise a pyranometer, a net radiometer, a quantum sensor, an actinometer, a bolometer, a thermopile, a photodiode, or any other known device for sensing broadband solar radiation flux density. Solar radiation sensor 520 may, in real-time or periodically, generate and communicate to processor 570 a signal indicative of a value of the sensed intensity of solar radiation (e.g., in watts per square meter) for storage in database 580 and/or transmission to worksite control system 108.

Atmospheric pressure sensor 530 may include any device (e.g., on fluid delivery machine 106 or positioned somewhere on worksite 100) configured to sense an atmospheric pressure of worksite 100. Atmospheric pressure sensor 530 may include a barometer sensor, such as a capacitive pressure sensor, an electromagnetic pressure sensor, a piezoresistive strain gauge pressure sensor, a piezoelectric pressure sensor, an optical pressure sensor, a potentiometric pressure sensor, or any other type of atmospheric pressure sensor known in the art. Atmospheric pressure sensor 530 may, in real-time or periodically, generate and communicate to processor 570 a signal indicative of a value of the sensed atmospheric pressure (e.g., in atms) for storage in database 580 and/or transmission to worksite control system 108.

Humidity sensor 540 may include any device (e.g., positioned on fluid delivery machine 106 or at a stationary location on or near worksite 100) configured to sense the humidity at worksite 100. For example, humidity sensor 540 may comprise an electric hygrometer, a hair tension hydrometer, a psychrometer, or any other device known in the art for sensing humidity. Humidity sensor 540 may, in real-time or periodically, generate and communicate to processor 570 a signal indicative of a value of the sensed humidity (e.g., in mass of water per unit volume of air) for storage in database 580 and/or transmission to worksite control system 108.

Wind sensor 550 may include any device (e.g., positioned on fluid delivery machine 106 or at a stationary location on or near worksite 100) configured to determine a speed and a direction of the wind on worksite 100. For example, wind sensor 550 may comprise a velocity anemometer, such as a laser Doppler anemometer, a sonic anemometer, a hot-wire anemometer, or a turbine anemometer; a pressure anemometer, such as a plate anemometer or a tube anemometer; or any other type of wind sensor known in the art. Wind sensor 550 may, in real-time or periodically, generate and communicate to processor 570 a signal indicative of values of the sensed wind speed and direction (e.g., 4 km/h NW) for storage in database 580 and/or transmission to worksite control system 108.

Precipitation sensor 560 may include any device (e.g., positioned on fluid delivery machine 106 or at a stationary location on or near worksite 100) configured to determine an amount or rate of precipitation on worksite 100. For example, precipitation sensor 560 may comprise a rain switch, a precipitation gauge, or any other type of precipitation-sensing device known in the art. Precipitation sensor 560 may, in real-time or periodically, generate and communicate to processor 570 a signal indicative of a value of the amount or rate of precipitation on worksite 100 for storage in database 580 and/or transmission to worksite control system 108.

The various components of mobile machines 102, fluid delivery machine 106, worksite control system 108, and weather station 112 may include an assembly of hardware, software, and/or firmware, including a memory, a central processing unit (“CPU”), and/or a user interface. Memory may include any type of RAM or ROM embodied in a non-transitory computer-readable storage medium, such as magnetic storage including floppy disk, hard disk, or magnetic tape; semiconductor storage such as solid state disk (SSD) or flash memory; optical disc storage; magneto-optical disc storage; or any other type of physical memory on which information or data readable by at least one processor may be stored. Singular terms, such as “memory” and “computer-readable storage medium,” may additionally refer to multiple structures, such a plurality of memories and/or computer-readable storage mediums. As referred to herein, a “memory” may comprise any type of computer-readable storage medium unless otherwise specified. A computer-readable storage medium may store instructions for execution by at least one processor, including instructions for causing the processor to perform steps or stages consistent with an embodiment herein. Additionally, one or more computer-readable storage mediums may be utilized in implementing a computer-implemented method. The term “computer-readable storage medium” should be understood to include tangible items and exclude carrier waves and transient signals. A CPU may include one or more processors for processing data according to a set of programmable instructions or software stored in the memory. The functions of each processor may be provided by a single dedicated processor or by a plurality of processors. Moreover, processors may include, without limitation, digital signal processor (DSP) hardware, or any other hardware capable of executing software. An optional user interface may include any type or combination of input/output devices, such as a display monitor, keyboard, and/or mouse.

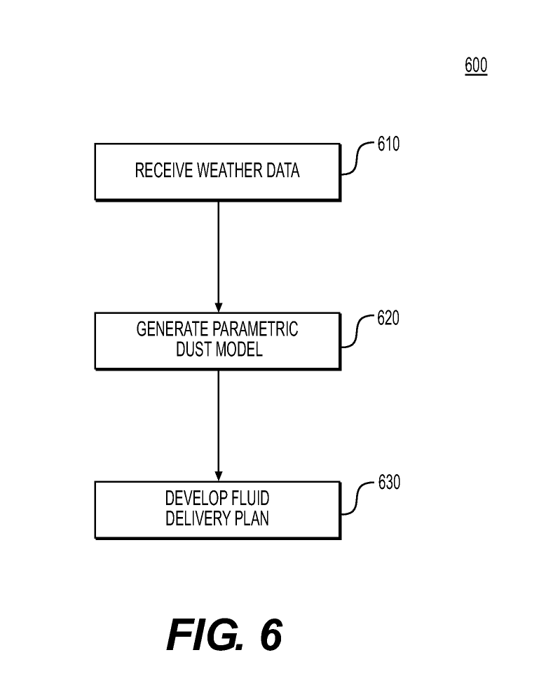

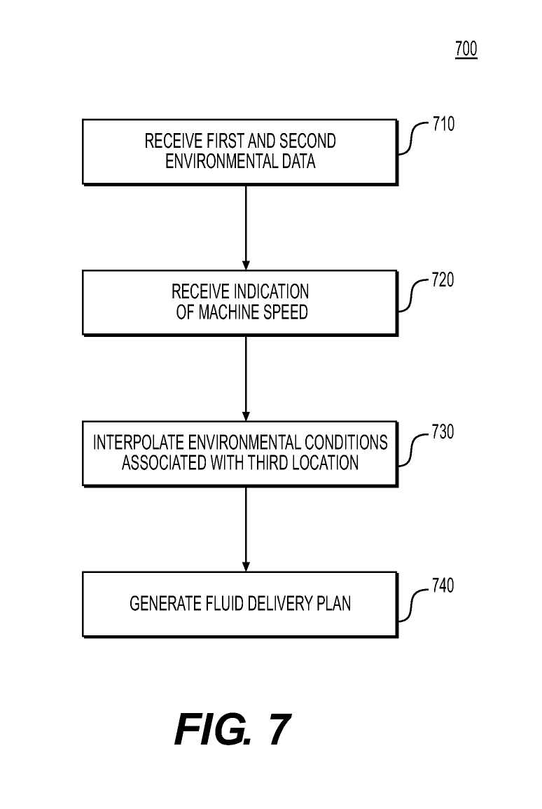

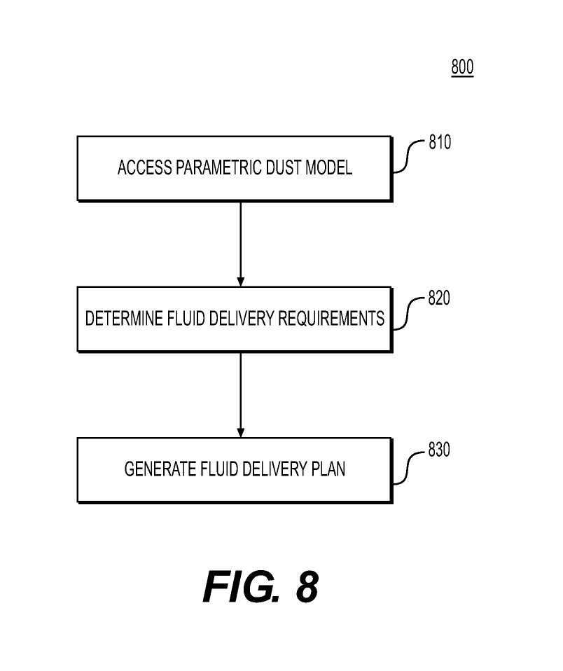

In accordance with certain embodiments, worksite control system 108 receives weather data from weather station 112, machine data from mobile machines 102, fluid delivery data from fluid delivery machines 106, and other data (e.g., traffic and weather data) from third-party sources. Worksite control system 108 stores and processes this data to analyze and interpolate environmental conditions (e.g., undue dust conditions), generate parametric dust models, determine fluid delivery requirements for locations within a worksite, and develop a fluid delivery plan to remedy undue dust conditions within the worksite. Worksite control system 108 may send the generated fluid delivery plan, or components thereof (e.g., fluid delivery routes), to one or more fluid delivery machines 106 for execution. FIG. 6, discussed below, provides further detail regarding techniques for identifying undue dust conditions. FIG. 7, discussed below, provides further detail regarding techniques for assessing environmental conditions. FIG. 8, discussed below, provides further detail regarding techniques for remedying undue dust conditions.

### INDUSTRIAL APPLICABILITY

The disclosed systems and methods for identifying undue dust conditions may be utilized to identify and remedy undue dust conditions. In particular, the disclosed systems and methods may analyze environmental and other data to develop a parametric dust model, which may be used to predict undue dust conditions. The disclosed systems and methods may also obtain real-time readings of dust conditions throughout a worksite and extrapolate the readings to determine the likely dust conditions at other areas in the worksite. Based on a comparison of the predicted and actual dust conditions throughout the worksite, the parametric dust model may be optimized and used to determine fluid delivery requirements and develop a fluid delivery plan to minimize undue dust conditions at the worksite. Unlike prior techniques for identifying or remedying undue dust conditions, which rely on operator observation or sensor measurements that show undue dust conditions have already begun to develop, the disclosed systems and methods may be used to predict and address undue dust conditions before they develop to a state that hinders worksite productivity.

FIG. 6 depicts an exemplary flow of a process 600 for identifying undue dust conditions, in accordance with an embodiment of the present disclosure. The steps associated with this exemplary process may be performed by or utilize the components of FIGS. 1-5. For example, the steps associated with the exemplary process of FIG. 6 may be performed by worksite control system 108 illustrated in FIG. 1. Moreover, data stored and processed by worksite control system 108 may be received from weather station 112 illustrated in FIGS. 1 and 5. Further, outputs may be sent from worksite control system 108 to fluid delivery machines 106 illustrated in FIGS. 1 and 2, components of which are described in further detail in FIGS. 3 and 4.

In step 610, weather data is received over a network. In one embodiment, weather data is received from a weather station (e.g., weather station 112) located on a worksite (e.g., worksite 100). In another embodiment, weather data is received from a third-party weather service. The received weather data may include ambient temperature, solar radiation intensity, atmospheric pressure, relative humidity, wind speed and direction, and/or an amount or rate of precipitation. Moreover, the received weather data may include current (i.e., real-time) weather data, historical weather data, or forecasted weather data. In one embodiment, current weather data may be provided by weather station 112, forecasted weather data may be provided by a third-party weather service, and historical weather data may be provided by weather station 112 or the third-party weather service.

In step 620, a parametric dust model is generated based on the received weather data. The parametric dust model may represent a forecast or prediction of the dust condition at locations throughout a worksite. In one embodiment, the received weather data is analyzed to determine whether current or forecasted weather conditions are likely to cause undue dust conditions at the worksite. For example, if the received weather data indicates that the solar radiation intensity is high, the relative humidity is low, the wind speed is high, and there has been little precipitation in the past week, then the parametric dust model may indicate that undue dust conditions are likely to develop in one or more locations throughout the worksite. If the received weather data indicates that there has been one inch of rain in the past twenty-four hours, then the parametric dust model may indicate that undue dust conditions are not likely to develop for at least twenty-four hours.

In one embodiment, machine data may be received over the network and used to generate the parametric dust model. For example, machine data may be received from machines (e.g., mobile machines 102) operating on a worksite. The received machine data may include an anti-lock braking system (ABS) status, a traction control system (TCS) status, a dynamic stability control (DSC) status, a dust measurement, a moisture value, and a geographic positioning system (GPS) location. In one embodiment, a machine may record and/or report its ABS status, TCS status, DSC status, dust measurement, moisture value, and GPS location periodically (e.g., once per minute), such that a record of the locations to which the machine traveled and the state of the machine (e.g., as reflected by the ABS, TCS, and DSC status indicators) and its environment (e.g., as reflected by the dust measurement and moisture value) at each of the locations may be provided for consideration in the generation of the parametric dust model. In an alternate embodiment, a machine may record and/or report its ABS status, TCS status, DSC status, dust measurement, and/or a moisture value, along with an associated GPS location, each time the ABS indicator, TCS indicator, or DSC indicator goes active.

An active ABS, TCS, or DSC status may indicate that the ground surface is so dry or so wet that the surface is slippery. Moreover, the moisture values represent the moisture level of the ground at the associated GPS location, which may affect, for example, the compactness of the soil or the slipperiness of the surface. The dust measurements represent the concentration of dust in the air at the associated GPS location. Thus, this data may be fed into the parametric dust model to affect the assessment of the likelihood that undue dust conditions exist at the various locations represented by the received machine data.

In one embodiment, traffic data is received over a network and used to generate the parametric dust model. For example, traffic data may be received over a network from a third-party mapping or traffic service. Alternatively, traffic data may be derived from machine data received over the network from machines operating on the worksite. For example, each machine may periodically (e.g., once per minute) report its location, acceleration, and velocity. This information may be used to determine the traffic conditions throughout the worksite.

The received traffic data may be used to determine the potential for undue dust conditions at various locations throughout the worksite. For example, high traffic areas may be more susceptible to undue dust conditions because heavy traffic may cause the soil (or other ground surface) to dry out. Moreover, heavy traffic over soil that is already dry is likely to stir up soil and create undue dust conditions that affect operator visibility.

In one embodiment, the received machine data and/or the received traffic data supplements the received weather data to form a parametric dust model. According to certain embodiments, certain data may be weighted more heavily than other data. For example, the received machine data may receive a higher weight than the received weather data in affecting the parametric dust model because the received data represents environmental conditions (e.g., moisture of soil or concentration of dust in the air) that are more determinative of the likelihood that undue dust conditions are present.

In one embodiment, the parametric dust model includes a plurality of geographic locations within a worksite and a dust condition associated with each geographic location. For example, the parametric dust model may identify ten locations within a worksite, along with a value representing the amount of dust particles (e.g., in micrograms) per cubic meter of air (or other unit of volume) at each location. This data may also be used to determine operator visibility at each location. Moreover, the parametric dust model may be used to determine the average speed of machines traveling the worksite or the average speed of machines traveling particular areas within the worksite.

In one embodiment, generating the parametric dust model may include processing certain data with a parameter estimator and providing the processed data as inputs into the parametric dust model. For example, the parameter estimator may process data that does not directly indicate the presence of undue dust conditions, but rather may be used to determine a likelihood that undue dust conditions exist or are likely to form. This data may include the received weather data; TCS, DSC, and ABS state data; and traffic data. For example, a low level of precipitation may indicate that a worksite is susceptible to undue dust conditions if other conditions are also present, but a low level of precipitation does not necessarily indicate that those conditions are already present or that they will necessarily develop.

Real-time environmental data may also be input into the parameter estimator, processed, and then forwarded as input into the prediction model. Real-time environmental data may include data that more closely correlates with the presence of an undue dust condition, such as data regarding the application of fluid to a worksite location by a fluid delivery machine, dust concentrations measured by a dust sensor, and moisture level of soil (or other ground surface) as measured by a moisture sensor. In addition to being input into the parameter estimator, which may affect the long-term modeling, this information may also be input directly into the parametric dust model to affect short-term modeling. For example, if fluid has just been applied to a location in the worksite, this should have a minimal effect on long-term forecasting of dust for the location, and thus should not greatly affect the long-term forecasted dust conditions for that location. A recent fluid delivery event should, however, more significantly affect the short-term dust conditions (and need for fluid) at the location. In one embodiment, if the parameter estimator has received insufficient data to generate the parametric dust model, a default parametric dust model may be utilized.

In one embodiment, the parametric dust model may be optimized based on real-time environmental data. Accordingly, real-time environmental data may be received over a network and compared to the generated parametric dust model. Based on this comparison, the generated parametric dust model may be adjusted. For example, the parametric dust model may indicate that the likely current or future dust concentration for a location is 50 μg/m³. If information in the received real-time environmental data (e.g., data from a dust sensor on a mobile machine operating on the worksite) indicates that the dust concentration at the location is 60 μg/m³, then the parametric dust model may be adjusted to reflect this difference. For example, the previous dust concentration associated with the location (i.e., 60 μg/m³) may be replaced with the most recent real-time observation (i.e., 50 μg/m³).

In step 630, a fluid delivery plan is developed based on the parametric dust model. In one embodiment, the fluid delivery plan identifies a plurality of geographic locations within a worksite and an amount of fluid to distribute per unit area to each geographic location. Further, in one embodiment, the water delivery plan may be sent over a network to at least one fluid delivery machine.

FIG. 7 depicts an exemplary flow of a process 700 for assessing environmental conditions, in accordance with an embodiment of the present disclosure. The steps associated with this exemplary process may be performed by or utilize the components of FIGS. 1-5. For example, the steps associated with the exemplary process of FIG. 7 may be performed by the worksite control system 108 illustrated in FIG. 1. Moreover, data processed by worksite control system 108 may be received from machines operating at a worksite, such as mobile machines 102. Further, outputs may be sent from worksite control system 108 to fluid delivery machines 106 illustrated in FIGS. 1 and 2, components of which are described in further detail in FIGS. 3 and 4.

In step 710, environmental data from a machine traveling through a first location and a second location is received. The environmental data may include first environmental data associated with the first location and second environmental data locating through a second location. For example, a machine (e.g., mobile machine 102) operating on a worksite may record environmental data as it travels through a worksite using one or more sensors located on the machine (e.g., dust sensor). In one embodiment, the environmental data includes dust measurements indicating the concentration of dust in the air surrounding the machine. The environmental data may also include a moisture value indicating the moisture level of the soil or other ground surface beneath the machine. Further, the environmental data may indicate the ground surface type (e.g., soil type) and slope.

In step 720, an indication of the speed the machine was traveling as it traveled through the first and second locations is received. In one embodiment, the machine provides both the speed it was traveling when it passed the first location and the speed it was traveling as it passed the second location. These speeds may be averaged to determine the average speed of the machine as it traveled between the first and second locations.

In step 730, environmental conditions associated with a third location are interpolated based on the first and second environmental data. In one embodiment, the third location is between the first and second locations. For example, a machine may periodically obtain environmental data as it travels throughout a worksite. In one embodiment, a dust sensor located on the machine may sense (i.e., sample) the dust conditions in the air surrounding the machine according to a preset sampling rate. The likely dust conditions in areas between the locations where the dust sensor performed sampling may be interpolated based on the data obtained at locations where sampling occurred.

In one embodiment, interpolating environmental conditions associated with a third location based on the first and second environmental data comprises identifying the third location based on the indication of the machine speed. For example, the location that the machine traveled through X seconds after it passed the first location may be determined based on the speed that the machine was traveling as it passed the first location. Alternatively, the location that the machine traveled through X seconds after it passed the first location may be determined based on the average speed that the machine traveled between the first and second locations. In yet another alternate embodiment, the location that the machine traveled through X seconds after it passed the first location may be determined based on the sampling rate of the sensor used to obtain the first and second environmental data. For example, if the sampling rate of the sensor is once per fifty seconds, then the machine should be one-fifth of the distance past the first location in the direction of the second location after ten seconds from having passed the first location.

In one embodiment, interpolating environmental conditions associated with a third location comprises applying a weighting function to the first and the second environmental data. If the third location is closer to the first location, the weighting function may weight the first environmental data more heavily than the second environmental data. If the third location is closer to the second location, the weighting function may weight the second environmental data more heavily than the first environmental data. For example, if the first and second locations are 100 meters apart and the third location is 25 meters from the first location and 75 meters from the second location, then the environmental conditions associated with the third location may be determined by weighting the first environmental by a factor of 0.75 and the second environmental data by a factor of 0.25. If the third location is the midpoint between the first and second locations, the first and second environmental data may be weighted equally.

In one embodiment, environmental data is received from more than two locations and used to interpolate the environmental conditions (e.g., dust conditions) at another location. For example, first, second, third, and fourth environmental data may be received from first, second, third, and fourth locations surrounding a fifth location and used to interpolate the environmental conditions at the fifth location. In one embodiment, a state estimator is used to determine the environmental conditions at a location from which environmental data has not been received. For example, a Kalman filter may be used to interpolate or determine the environmental conditions at the location based on environmental data received from a plurality of other locations.

In step 740, a fluid delivery plan may be generated based on the first environmental data, second environmental data, and interpolated environmental conditions. The fluid delivery plan may identify an amount of fluid (e.g., water and/or other dust suppressant) to be distributed to each of the first, second, and third locations based on the first environmental data, second environmental data, and interpolated environmental conditions. In one embodiment, the fluid delivery plan may include a schedule for delivering fluid to the first, second, and third locations. The fluid delivery plan may also indicate a route for a fluid delivery machine to follow in order to deliver fluid to the first, second, and third locations. In one embodiment, the fluid delivery plan is sent over a network to one or more fluid delivery machines for execution. Fluid delivery plans are described in more detail in the description of FIG. 8 below.

FIG. 8 depicts an exemplary flow of a process 800 for remedying undue dust conditions, in accordance with an embodiment of the present disclosure. The steps associated with this exemplary process may be performed by or utilize the components of FIGS. 1-5. For example, the steps associated with the exemplary process of FIG. 8 may be performed by worksite control system 108 illustrated in FIG. 1. Moreover, data processed by worksite control system 108 may be received from weather station 112 and machines operating on a worksite (e.g., mobile machines 102 or fluid delivery machines 106). Further, outputs may be sent from worksite control system 108 to fluid delivery machines 106 illustrated in FIGS. 1 and 2, components of which are described in further detail in FIGS. 3 and 4.

In step 810, a parametric dust model is accessed. In one embodiment, the parametric dust model may be the parametric dust model generated in step 620 of FIG. 6. For example, the parametric dust model may be generated based on weather data received from weather station 112 or a third-party weather service. The parametric dust model may further be generated based on data received from machines (e.g., mobile machines 102) operating on a worksite, including an ABS state, TCS state, DSC state, moisture value, or dust measurement. Moreover, the parametric dust model may be generated based on traffic data from the worksite, which may be received from a third-party mapping or traffic service or determined based on geolocation and/or speed information received from machines operating on the worksite.

The parametric dust model may comprise a plurality of locations in a worksite and a dust measurement associated with each location. In one embodiment, the dust measurement may represent a concentration of dust in the air at the location. In another embodiment, the dust measurement may represent conditions of the ground surface, such as the moisture value of the soil or other surface.

In step 820, a fluid delivery requirement is determined for each of the plurality of locations based on the parametric dust model. For example, the dust measurement for a location may be compared to a threshold value. If the dust measurement exceeds the threshold value, then it may be necessary to delivery fluid to the location to remedy the dust conditions at the location. In one embodiment, determining the fluid delivery requirement for a location may comprise determining an amount of fluid to deliver to the location. For example, the amount of fluid needed at a location may increase proportionally with the dust measurement.

In one embodiment, real-time environmental data is received from at least one machine operating in the worksite. For example, data describing current machine conditions or environmental conditions may be received from a machine while it is operating on the worksite or shortly thereafter. The real-time environmental data may include an ABS state, TCS state, DSC state, moisture value, or dust measurement. As discussed above, the ABS state, TCS state, and DSC state may be active when certain road conditions exist affecting the traction of the machine. The moisture value and dust measurement may be determined by a moisture sensor and dust sensor, respectively, on the machine.

The real-time environmental data may be used to determine the fluid delivery requirement for each (or a subset) of the plurality of locations represented in the parametric dust model. For example, the real-time environmental data may be used to determine the fluid delivery requirement for the locations from which the real-time environmental data was collected. In one embodiment, the fluid delivery requirement for a location from which real-time data has recently been received may be affected more by the real-time environmental data than the parametric dust model, as the real-time environmental data is more likely to represent the current need for fluid at the location. Accordingly, the real-time environmental data may be weighted based on the recency of the data. Moreover, certain types of real-time environmental data (e.g., dust measurements) may be weighted more heavily than other types (e.g. ABS, DSC, TCS state) because those types may be more directly indicative of the dust conditions of a location.

In step 830, a fluid delivery plan is generated based on the determined fluid delivery requirements. The fluid delivery plan describes how fluid may be delivered throughout a worksite to remedy undue dust conditions at one or more locations within the worksite. In one embodiment, the fluid delivery plan comprises a route for delivering fluid to locations in the worksite using a fluid delivery machine. In one embodiment, the fluid delivered to the locations may be water. Other dust-suppressing fluids may be used as an alternative to, or in addition to, water to remedy undue dust conditions, as would be understood by one or ordinary skill in the art.

In one embodiment, the route includes a sequence according to which the locations within the worksite should be visited. In one embodiment, the route and sequence may be determined based on a need to visit all locations in the worksite that have undue dust conditions. Thus, the determined sequence for visiting the locations may be the sequence that allows fluid to be delivered to all locations experiencing undue dust conditions in the least amount of time (or using the least amount of fuel or fluid). This may be advantageous where there are a sufficient number of fluid delivery machines and/or a sufficient capacity of fluid and/or fuel per water delivery machine to cover every location experiencing undue dust conditions.

In another embodiment, the route and sequence may be determined based on a priority or need for visiting each location. For example, the locations with the highest concentrations of dust may be included at the beginning of the sequence and followed by locations with lower concentrations of dust (e.g., organized by descending level of dust). This may be advantageous where there are a high number of locations with undue dust conditions and/or an insufficient number of fluid delivery machines (or fuel/fluid) to deliver the fluid in one trip.

In one embodiment, the fluid delivery plan specifies a rate of flow for the fluid and a speed at which the fluid delivery machine should travel the route. The rate of flow may vary throughout the route based on the fluid delivery requirements of different locations along the route and/or the speed that the fluid delivery machine is traveling throughout the route. For example, the rate of flow may be greater where the fluid delivery requirements are greater. The rate of flow may be lower where the fluid delivery requirements are lower or where the machine must travel at a lower speed based, for example, on the terrain (e.g., unstable terrain, hills). Alternatively, the rate of flow may be a static rate determined based on the average fluid delivery requirements of locations throughout the worksite and/or the speed of the machine as it travels the route.

In one embodiment, the fluid delivery plan may specify one or more spray nozzle settings to be used along the route. These settings may vary by location along the route. The optimal spray nozzle setting may be determined and specified based on the size of the location experiencing undue dust conditions, the available amount of fluid for delivery in a single pass (i.e., a single traversal of the route by a single fluid delivery machine), the number of fluid delivery machines available for delivering fluid, and the number of times the fluid delivery machine will traverse the route.

In one embodiment, the fluid delivery plan may indicate the locations along the route at which the speed of the fluid delivery machine, the spray nozzle setting, or the rate of flow should change. Thus, any one of these factors may change once the fluid delivery machine detects that it has reached a location that has different fluid delivery requirements. In another embodiment, the fluid delivery plan may indicate when these changes should occur based on the elapsed time in the route. For example, the route may include a schedule according to which the fluid delivery machine should visit each location in the route. Thus, the speed of the machine, spray nozzle setting, or rate of flow may change based on the amount of time that has elapsed since the fluid delivery machine began traversing the route.

In one embodiment, generating a fluid delivery plan may comprise determining a volume of fluid stored by a fluid delivery machine. The fluid delivery route may be optimized based on the stored volume of fluid. For example, if the stored volume of fluid is less than the amount of fluid needed to meet the fluid delivery requirements of the worksite, then the fluid delivery plan may be optimized to focus on delivering fluid to those locations with the highest dust measurements.

In one embodiment, generating a fluid delivery plan comprises determining an amount of fuel stored by the fluid delivery machine. Alternatively, determining a fluid delivery plan may comprise determining a fuel capacity of the fluid delivery machine. The fluid delivery route may be optimized based on the stored amount of fuel (or based on the fuel capacity of the fluid delivery machine). For example, if the stored amount of fuel (or fuel capacity) is insufficient to fuel the fluid delivery machine for the full distance of the route, then the fluid delivery plan may be optimized to focus on delivering fluid to those locations with the highest dust measurements. In one embodiment, the route may be determined based on the location of fuel stations within or near the worksite.

In one embodiment, the fluid delivery plan may contemplate fluid delivery by multiple fluid delivery machines. Accordingly, generating the fluid delivery plan may include determining the availability of multiple fluid delivery machines and determining a route for each available fluid delivery machine. Moreover, each route may be determined based on the fuel and fluid capacities of the available fluid delivery machines. The one or more routes included in the fluid delivery plan may be sent over a network to the one or more fluid delivery machines assigned to the routes.

In one embodiment, the fluid delivery machine records and reports data regarding the actual delivery of fluid while on a route, including the amount of fluid delivered and associated location information. This fluid delivery data may be received from the fluid delivery machine and used to adjust the fluid delivery requirement for at least one of the plurality of locations along the route. For example, the fluid delivery requirement for a location may be reduced based on confirmation from the fluid delivery machine that fluid was delivered to the location. In one embodiment, the fluid delivery requirement for the location may be reduced by the amount of fluid delivered to the location. A revised fluid delivery plan may be generated reflecting the adjusted fluid delivery requirement for one or more locations. The revised fluid delivery plan may be used to train the fluid delivery machine, such that the fluid delivery machine delivers fluids more efficiently on future routes.

In one embodiment, the fluid delivery machine may be followed by a trailing machine along the fluid delivery route. The trailing machine may include a TCS indicator, a DSC indicator, an ABS indicator, a dust sensor, and a moisture sensor. The trailing machine may record each instance in which the TCS indicator, DSC indicator, or ABS indicator goes active, along with an associated location. The trailing machine may also record dust measurements using the dust sensor and moisture values using the moisture sensor. The states of the TCS, DSC, and ABS indicators and the values recorded by the dust sensor and moisture sensor may be reported (e.g., to the worksite control system) periodically (e.g., once per minute) or based upon the occurrence of a specified condition (e.g., indicator goes active, dust measurement exceeds threshold, moisture value exceeds threshold).

In one embodiment, the trailing machine data is received and used to adjust the fluid delivery requirement for at least one of the plurality of locations in the fluid delivery plan. For example, if the trailing machine data indicates that undue dust conditions associated with a location have subsided since the fluid delivery machine delivered fluid to the location, the fluid delivery requirement for that location may be lowered (or reduced to zero). If the trailing machine data indicates that undue dust conditions associated with a location persist even after the fluid delivery machine delivered fluid to the location, the fluid delivery requirement for that location may remain the same, be increased, or be lowered, depending on precise dust conditions at the location. A revised fluid delivery plan may be generated reflecting the adjusted fluid delivery requirement for one or more locations. The revised fluid delivery plan may be used to guide fluid delivery machines on subsequent routes through the worksite and may include the same or different routes, fluid delivery machines, and other details (e.g., speed of fluid delivery machines, flow of fluid, spray nozzle settings).

Several advantages over the prior art may be associated with the disclosed systems and methods for identifying and remedying undue dust conditions. Unlike the techniques described in the prior art, the disclosed techniques for identifying and remedying undue dust conditions may predict and remedy undue dust conditions before they elevate to a level that hinders the ability of operators to perform work on a worksite. The disclosed techniques also enable more discrete assessment of environmental conditions throughout a worksite based on interpolation of environmental data. Moreover, the disclosed techniques may more efficiently remedy undue dust conditions by using optimized fluid delivery plans.

It will be apparent to those skilled in the art that various modifications and variations can be made to the disclosed systems and methods for identifying and remedying undue dust conditions. Other embodiments will be apparent to those skilled in the art from consideration of the specification and practice of the disclosed systems and methods for identifying and remedying undue dust conditions. It is intended that the specification and examples be considered as exemplary only, with a true scope being indicated by the following claims and their equivalents.

What is claimed is:

1. A method for assessing environmental conditions, the method comprising the following steps performed by one or more processors:
receiving environmental data from a machine traveling through a first location and a second location, the environmental data comprising first environmental data associated with the first location and second environmental data associated with the second location; and
interpolating environmental conditions associated with a third location based on the first and second environmental data.

2. The method of claim 1, wherein the third location is between the first and second locations.

3. The method of claim 1, wherein:
the first and second environmental data comprise dust measurements; and
interpolating environmental conditions associated with a third location based on the first and second environmental data comprises interpolating a dust condition associated with the third location.

4. The method of claim 1, further comprising:
receiving an indication of the speed the machine was traveling as it traveled through the first and second locations,
wherein interpolating environmental conditions associated with the third location comprises identifying the third location based on the indication of the machine speed.

5. The method of claim 1, wherein interpolating environmental conditions associated with a third location comprises applying a weighting function to the first and second environmental data.

6. The method of claim 5, wherein the weighting function weights the first environmental data more heavily if the third location is closer to the first location than the second location and weights the second environmental data more heavily if the third location is closer to the second location than the first location.

7. The method of claim 1, further comprising generating a fluid delivery plan based on the first environmental data, second environmental data, and interpolated environmental conditions.

8. A non-transitory computer-readable storage medium storing instructions for assessing environmental conditions, the instructions causing at least one processor to perform operations comprising:
receiving environmental data from a machine traveling through a first location and a second location, the environmental data comprising first environmental data associated with the first location and second environmental data associated with the second location; and
interpolating environmental conditions associated with a third location based on the first and second environmental data.

9. The non-transitory computer-readable storage medium of claim 8, wherein the third location is between the first and second locations.

10. The non-transitory computer-readable storage medium of claim 8, wherein:
the first and second environmental data comprise dust measurements; and
the instructions cause the at least one processor to interpolate environmental conditions associated with a third location based on the first and second environmental data by interpolating a dust condition associated with the third location.

11. The non-transitory computer-readable storage medium of claim 8, wherein the instructions cause the at least one processor to:
receive an indication of the speed the machine was traveling as it traveled through the first and second locations,
wherein interpolating environmental conditions associated with the third location comprises identifying the third location based on the indication of the machine speed.

12. The non-transitory computer-readable storage medium of claim 8, wherein the instructions cause the at least one processor to interpolate environmental conditions associated with a third location by applying a weighting function to the first and second environmental data

13. The non-transitory computer-readable storage medium of claim 8, wherein the instructions cause the at least one processor to generate a fluid delivery plan based on the first environmental data, second environmental data, and interpolated environmental conditions.

14. A system for assessing environmental conditions, comprising:
a memory that stores a set of instructions; and
at least one processor in communication with the memory and configured to execute the set of instructions to:
receive environmental data from a machine traveling through a first location and a second location, the environmental data comprising first environmental data associated with the first location and second environmental data associated with the second location; and
interpolate environmental conditions associated with a third location based on the first and second environmental data.

15. The system of claim 14, wherein the third location is between the first and second locations.

16. The system of claim 14, wherein:
the first and second environmental data comprise dust measurements; and
the at least one processor is further configured to interpolate environmental conditions associated with a third location based on the first and second environmental data by interpolating a dust condition associated with the third location.

17. The system of claim 14, wherein the at least one processor is further configured to:
receive an indication of the speed the machine was traveling as it traveled through the first and second locations,
wherein interpolating environmental conditions associated with the third location comprises identifying the third location based on the indication of the machine speed.

18. The system of claim 14, wherein the at least one processor is further configured to interpolate environmental conditions associated with a third location by applying a weighting function to the first and second environmental data.

19. The system of claim 18, wherein the weighting function weights the first environmental data more heavily if the third location is closer to the first location than the second location and weights the second environmental data more heavily if the third location is closer to the second location than the first location.

20. The system of claim 14, wherein the at least one processor is further configured to generate a fluid delivery plan based on the first environmental data, second environmental data, and interpolated environmental conditions.
